(19) United States
(12) Patent Application Publication
(10) Pub. No.: US 2022/0079961 A1
(43) Pub. Date: Mar. 17, 2022

(54) NASAL SPRAY FORMULATIONS USING BOTANICALS, STEROIDS ORGANOSILANE QUATERNARIES, POLYOL STABILIZING AGENTS AND NONIONIC SURFACTANT AS ANTIMICROBIALS, ANTIVIRALS AND BIOCIDES TO PROTECT THE CELLS, SKIN AND HAIR OF NASAL PASSAGES

(71) Applicant: AP Goldshield LLC, Locust Valley, NY (US)

(72) Inventor: Thomas L. Higgins, Locust Valley, NY (US)

(21) Appl. No.: 17/476,768

(22) Filed: Sep. 16, 2021

Related U.S. Application Data
(60) Provisional application No. 63/079,293, filed on Sep. 16, 2020.

(51) Int. Cl. A61K 31/695; A61K 31/05; A61K 31/573; A61K 9/00

(52) U.S. Cl. CPC A61K 31/695; A61K 9/0043; A61K 31/573; A61K 31/05

(57) ABSTRACT

This invention relates to the formulations and methods for use to sanitize and protect goblet secretory cells, which produce mucus, as well as the skin cells and hair within the nasal passage to inhibit bacterial, fungal and viral infections. A novel nasal spray composition of the invention includes an organosilane compound, C₂₃H₅₂ClNO₃Si cross-linked to a botanical, Thymol, C₁₀H₁₄O, a steroid, dexamethasone, C₂₂H₂₉FO₅, a polyol stabilizing agent and a nonionic surfactant that adheres to mucosa, skin cells and hair in the nasal passages. The composition creating a 12-24 hour, and up to a 48-hour bio-barrier to inhibit an infection from bacteria fungi, and viruses.

### CROSS REFERENCE TO RELATED APPLICATIONS

This application claims priority of U.S. Provisional Application No. 63/079,293 filed on Sep. 16, 2020 and incorporates the entirety of its disclosure by reference into this application.

### BACKGROUND OF THE INVENTION

### 1. Field of the Invention

This invention relates to the field of compositions and methods to sanitize and protect nasal passages from viral inhalation and infection. The invention relates to compositions employing a botanical compound with the formula C₁₀H₁₄O (Thymol), and organosilane compounds, and their use as biocidal agents with residual-performing killing functionalities against microscopic organisms such as bacteria, fungi, and viruses.

The invention further relates to the field of composition and methods to sanitize nasal passages and to employ a steroid, dexamethasone, with the formula C₂₂H₂₉FO₅ to reduce deaths caused by coronavirus. This tri-functional formula of a botanical, a steroid and residual performing organosilane to prevent coronavirus in the nasal passage from attaching to the body's cell receptors and to inhibit deaths by the employment of a steroid with the formula, C₂₂H₂₉FO₅.

### 2. Background of the Related Art

With the rise of “superbugs”, MRSA, Staph, HlNl (“Swine-flu”), and the spreading of pandemics, such as SARS and COVID19, there is a strong awareness in the public's mind about infections and their personal safety. Institutions, ranging from nursing homes to hospitals, schools to daycare centers, commercial entities to cruise lines are increasingly aware of the huge liability that these sorts of infections represent. There are literally thousands of well-documented cases of patients entering a hospital for a relatively minor procedure, becoming infected and either enduring a long painful recovering process, or even worse, dying. With the release of Covid19 and its global negative impact, with 8.5 million plus cases and 450,000 plus deaths as of June 2020, there is a need for technologies that can mitigate the infection and the spreading of this disease and other viral pathogens.

The rise of these tenacious microbes created a demand for a product that reduces the probability of contracting and spreading these infections by simply reducing exposure to viral pathogens, and protecting against reinfection.

A new study by researchers from the Massachusetts Institute of Technology (MIT) and Harvard University, both in Cambridge, Mass.—alongside colleagues from other institutions—may have pinpointed the cells that SARS-CoV-2 primarily targets in the human body.

The study—published in the journal Cell 181, 1016-1035. E1-e11 (May 28, 2020)-built on existing investigations showing that SARS-CoV-2 uses two proteins that act as receptors to invade host cells and replicate. These proteins are angiotensin-converting enzyme 2 (ACE2) and transmembrane protease serine 2 (TMPRSS2). The researchers wanted to find out which cells in the human body express these proteins, and they focused on sets of cells in the nasal passages, lungs, and small intestine. This was because people with COVID-19 most often experience respiratory and digestive symptoms.

“As soon as we realized that the role of these proteins had been biochemically confirmed,” says study co-author Jose Ordovas-Montanes, Ph.D., “we started looking to see where those genes were in our existing datasets.” “We were really in a good position to start to investigate which are the cells that this virus might actually target,” he adds. Cell 2020 May 28; 181(5):1016-1035.e19. doi: 10.1016/j.cell.2020.04.035. Epub 2020 Apr. 27.
“Our goal is to get information out to the community and to share data as soon as is humanly possible, so that we can help accelerate ongoing efforts in the scientific and medical communities,” adds study co-author Prof. Alex Shalek, from MIT. Cell2020 May 28; 181(5):1016-1035.e19. doi: 10.1016/j.cell.2020.04.035. Epub 2020 Apr. 27.

The researchers found that in the nasal passages, “goblet secretory cells”—which produce mucus—express RNA that allows them to produce both ACE2 and TMPRSS2, the coronavirus uses to infect and invade cells. In lung tissue, they settled on type II pneumocytes, which are the cells that line the alveoli (air sacs) and allow them to stay open. TMPRSS2 activates the coronavirus spike protein aiding the entry of the virus into human cell. Due to the combined binding and activation process, the virus finds its way into the cells.

In the lungs, the team found that the RNAs for the two proteins were seen in Type II pneumocytes, which line the air sacs or alveoli, the functional unit if the respiratory tract where gas exchange happens. The team also found that the cells called absorptive enterocytes in the intestines, which play a vital role in the absorption of nutrients, also express the RNAs for the two proteins.

The efficacy of the botanical compound with the formula C₁₀H₁₄O (Thymol) has been recognized for years as an effective composition in treating viral contamination. It fails, however, to provide any residual action and an effective vehicle for delivery of this compound to attach to affected cells has not been disclosed.

Organosilanes containing quaternary ammonium halides and hydrolysable alkoxy groups bonded to silicon have been employed in a wide variety of applications. The hydrolysable groups enable these compounds to form bonds to substrates that contain hydroxyl, alkoxy, oxide, and similar reactive moieties. Organosilanes have been used in skin sanitizers to attach to the carbohydrates of skin that polyhydroxylated providing a residual action of protection.

Organosilanes are employed in hand rinse formulations, to bond to skin, nail, and hair for reducing or eliminating microorganisms that may be present on the area to which the formulation is applied.

The efficacy of organosilanes as antimicrobial agents has been recognized for decades. The type or organosilane commonly used as antimicrobial agents have the general formula RnSiX4−n, where n ranges from 0 to 3, R is a nonhydrolyzable organic group, and X is a hydrolysable group. In U.S. Pat. No. 5,959,014 (the '014 patent, which is incorporated by reference in its entirety) the inventors, Liebeskind et al., recognized that commercially available organosilanes of this type often suffer from self-condensation in water, which limits the time aqueous solutions of the compound can be stored. The '014 patent teaches the reaction of an organosilane with a polyol containing at least three hydroxy groups, any two of the three hydroxy groups separated by at least three intervening atoms. By treating a substrate with the resultant product, the substrate is rendered antimicrobial and effective against bacteria and certain fungi.

Liebeskind et al. further disclose in U.S. Pat. No. 6,221,944 (the '944 patent, which is incorporated by reference in its entirety) the addition of a polyol having at least three hydroxy groups, all of the three hydroxy groups separated by at least three intervening atoms, that bonds to the silicon atom of the organosilane by removing a hydrogen atom of at least one of the hydroxy groups of the polyol; this leaves the oxygen atom to bond to the silicon.

Recognizing that, while organosilane-based water-soluble antimicrobial compositions have advanced significantly in recent years, there remain challenges in achieving long-lasting broad-spectrum antimicrobial activity on skin. It had been proposed that the reaction of these chemistries provide a durable, long-lasting broad-spectrum killing functionality.

Thus, Higgins and Shlisky describe the employment of non-ionic agents for organosilane-based water-soluble antimicrobial compositions, along with methods of making and using them, in U.S. Pat. No. 9,089,138 to Higgins and Shlisky (hereinafter “Higgins-Shlisky” and which is herein incorporated by reference as if fully set forth in its entirety). The use of a specific concentration of non-ionic agents reduces the surface energy (surface tension) between the solution and the substrate, allowing for better and faster penetration of the antimicrobial composition, and allowing a stronger siloxane bond between the antimicrobial and the substrate to form. They also reduce oils and other foreign materials on skin providing faster attachment of the antimicrobial to skin, hair and nails.

In published International Patent Application, WO 2011/123623 (which is incorporated by reference in its entirety), Higgins et al., described the use of organosilanes quaternaries, in combination with benzalkonium chloride, and methyl anthranilate in a water based system that could be applied to human skin, nails, and hair as a topical antimicrobial formulation that will attach to the carbohydrates of skin which polyhydroxylate providing longer lasting skin protection.

In studies at Oxford University by Martin Landray, professor and co-leading the trail on dexamethasone found this steroidal compound reduced death rates by about one third; The RECOVERY Collaborative Group, DEXAMETHASONE IN HOSPITALIZED PATIENTS WITH COVID-19_PRELIMINARY REPORT; New England Journal of Medicine (Jul. 17, 2020). However, a vehicle for effective delivery of this compound is not disclosed.

Accordingly, there remains an urgent need for an effective anti-viral formulation that reduces the probability of contracting and spreading these emerging viral infections by simply reducing exposure to the virus and protecting against reinfection.

### SUMMARY OF THE INVENTION

A formulation is disclosed that chemically links (reacts) botanical compounds of formula C₁₀H₁₄O with an organosilane-based water-soluble antimicrobial composition that retains its residual antimicrobial activity even after it kills (sterilizes/disinfects) any existing infectious bacteria, fungi, and virus. The targets may include disease organisms, including dangerous viruses, such as, Covid 19; SARS-CoV-2 attributed to causing severe respiratory infections, pneumonia, and death.

In some embodiments, the organosilane may preferably have the formula C₂₃H₅₂ClNO₃Si. In some embodiments the means of dispensing of this invention may include, but will not be limited to, oral or nasal sprays, aerosols, eye drops and solidified into lozenges. In some embodiments the invention provides a nasal anti-viral formulation that provides residual disinfecting nasal spray action. In some embodiments the invention is incorporated in a formulation to inhibit further viral isolates from spreading to the throat and lungs.

In some embodiments the invention provides compositions and methods to sanitize and protect nasal passages from viral inhalation and infection. The compositions employ a botanical compound with the formula C₁₀H₁₄O (Thymol), and organosilane compounds are used as biocidal agents with residual-performing killing functionalities against microscopic organisms such as bacteria, fungi, and viruses.

In some embodiments, the invention provides compositions and methods to sanitize nasal passages and to employ a steroid, dexamethasone, with the formula C₂₂H₂₉FO₅ to reduce deaths caused by coronavirus. The invention's tri-functional formula embodying a botanical, a steroid and a residual performing organosilane that operate to prevent coronavirus in the nasal passage from attaching to the body's cell receptors and to inhibit deaths by the employment of a steroid with the formula, C₂₂H₂₉FO₅.

In some embodiments the formulation may be supplemented with other medicinal agents, such as dexamethasone or numbing agents, such as a local anesthetic, including but not limited to menthol, hydrocodone, lidocaine, tetrahydrozoline.

This, being a summary, is necessarily brief and does not put forth all of the features and advantages of the novel formulation, its method of making, or its use in its applications. The invention may be more fully understood with reference to the drawings and the detailed description that follow.

### BRIEF DESCRIPTION OF THE DRAWINGS

FIG. 1 is a schematic depiction of the way the residual-performing biocidal formulation of the invention bonds to the surface or skin of a keratinized mammalian epithelial cell.

FIG. 2 depicts the residual-performing biocidal formulation of the invention attacking a pathogen.

FIG. 3 is a table providing a summary of the Higgins-Shlisky Organosilane formulation's safety on Normal Human lung cells, and Human non-small cell lung Tumor cells at various concentrations of the organosilane active ingredient.

### DETAILED DESCRIPTION OF INVENTION

To satisfy the urgent need for an effective anti-viral formulation that reduces the probability of contracting and spreading these emerging viral infections, the present invention reduces exposure to the virus and protects the user/host against reinfection.

Accordingly, this invention provides a formulation that chemically links (reacts) botanical compounds of formula C₁₀H₁₄O with an organosilane-based water-soluble antimicrobial composition that retains its residual antimicrobial activity even after it kills (sterilizes/disinfects) any existing infectious bacteria, fungi, and virus. The targets may include disease organisms, including dangerous viruses, such as, Covid 19; SARS-CoV-2 attributed to causing severe respiratory infections, pneumonia, and death.

The composition of the invention creates a bio-barrier to bacteria, fungi and viruses by attaching to the goblet secretory cells which produce mucus, skin cells, hair within the nasal passage to inhibit infection. The goblet secretory cells produce both ACE2 and TMPRSS2 in lung tissue, such as type II pneumocytes, which are the cells that line the alveoli (air sacs) and allow them to stay open. As known, TMPRSS2 activates the coronavirus spike protein aiding the entry of the virus into human cell, and due to the combined binding and activation process, the virus finds its way into the cells. The inventive composition mitigates these reactions by inhibiting the virus from taking effect within the nasal passage and neutralizes the virus within the release of the protein in mucus.

In some embodiments, the organosilane of the invention may preferably have the formula C₂₃H₅₂ClNO₃Si. In some embodiments, the means of dispensing of this invention may include, but will not be limited to, oral or nasal sprays, aerosols and solidified into lozenges. In some embodiments, the invention provides a nasal anti-viral formulation that provides residual disinfecting nasal spray action. In some embodiments, the invention is incorporated in a formulation to inhibit further viral isolates from spreading to the throat and lungs.

In some embodiments, the invention provides compositions and methods to sanitize and protect nasal passages from viral inhalation and infection. The compositions employ a botanical compound with the formula C₁₀H₁₄O (Thymol) and organosilane compounds, for use as biocidal agents with residual-performing killing functionalities against microscopic organisms such as bacteria, fungi, and viruses.

In some embodiments, the invention provides composition and methods to sanitize nasal passages and to employ a steroid, dexamethasone, with the formula C₂₂H₂₉FO₅ to reduce deaths caused by coronavirus. The invention's, tri-functional formula of a botanical, a steroid and residual performing organosilane to prevent coronavirus in the nasal passage from attaching to the body's cell receptors and to inhibit deaths by the employment of a steroid with the formula, C₂₂H₂₉FO₅.

In some embodiments, the formulation may be supplemented with other medicinal agents, such as dexamethasone or numbing agents, such as a local anesthetic, including but not limited to menthol, hydrocodone and/or lidocaine.

The reaction of the organosilanes, preferably trihydroxysilylpropyldimethyloctadecyl ammonium chloride (IUPAC name 1-Octadecanaminium, N,N-dimethyl-N-(3-(trihydroxysilyl)propyl) chloride) of the formula C₂₃H₅₂ClNO₃Si, at 0.5% to 2.0%, and the botanical, Thymol of the formula C₁₀H₁₄O, may form a residual-performing biocidal formulation. The residual-performing biocidal formulation attaches to the carbohydrates of skin that polyhydroxylate. The formulation adheres to surfaces of mucosa, including oral and nasal mucosa and airways, hair such as nose hair, and nails, and creates a bio-barrier on all these that have keratinized epithelial cells on the surface to which the formula adheres.

The addition of a steroid, dexamethasone, with the formula C₂₂H₂₉FO₅ cross linked to the carbon chain of the organosilane has the added effects of reducing infections caused by the viral proteins in Covid-19 and SARS-CoV-2, as they use two proteins that act as receptors to invade host cells and replicate (as mentioned above). These proteins are angiotensin-converting enzyme 2 (ACE2) and transmembrane protease serine 2 (TMPRSS2). By identifying the cells in the human body that express these proteins, such as the sets of cells in the nasal passages, lungs, and small intestine, the steroid in the formula composition to directly attack these proteins.

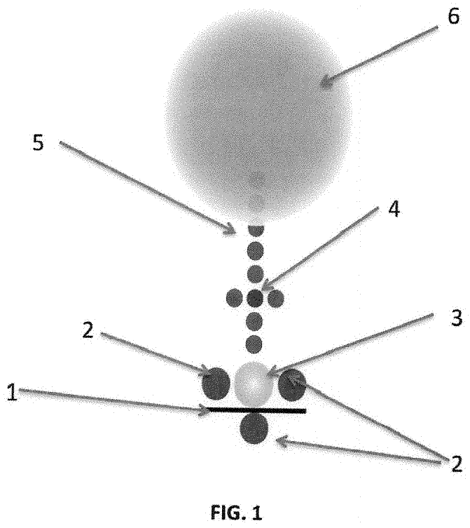

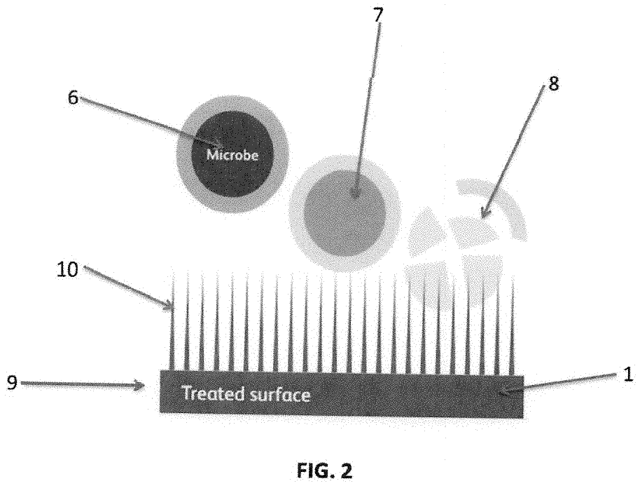

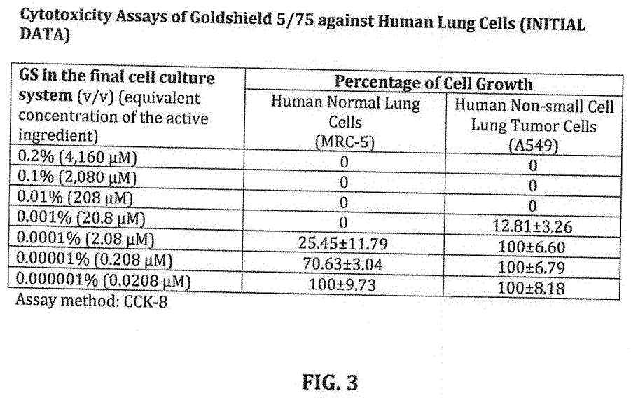

FIG. 1 is a schematic depiction of the way the residual-performing biocidal formulation of the invention bonds to the surface or skin of a keratinized mammalian epithelial cell. FIG. 2 depicts the residual-performing biocidal formulation of the invention attacking a pathogen. FIG. 3 is a table providing a summary of the Higgins-Shlisky Organosilane formulation's safety on Normal Human lung cells, and Human non-small cell lung Tumor cells at various concentrations of the organosilane active ingredient.

### Example

A critical issue in the development of any therapeutic is the safety issue required to eventually conduct human trials, before assessing the viability, the effectiveness, the safety and repercussions of its use in these human trials. This invention is a work in process to get to the point of human trials. A predicate to get there is the safety in vivo assessment on human cells to identify any cytotoxicity issues. Described below is a study conducted by Brigham Young University's by Bradford K. Berges, Ph.D., Associate Professor for Microbiology and Molecular Biology Brigham Young University. It demonstrates that the formulation of this invention at its full strength and diluted strength has the potential for human trials, the ultimate predicate for eventual regulatory approval.

TABLE 1
Cytotoxicity Assays of Goldshield LLC Product
against Human Lung and Colon Cells

| | Percentage of Cell Growth | |
|---|---|---|
| | Primary Human Lung | Human Colorectal |
| | Cells | HRT-18G Cells |
| | (ATCC #PCS-300-010) | (ATCC #CRL-11663) |
| 15% dilution of | Completely healthy | Completely healthy |
| full strength | | |
| Full strength | Completely healthy | Completely healthy |

Background:

AP GoldShield LLC (Referred to as “Goldshield LLC”) provided a product that they requested to be tested in order to determine if there were any potential safety concerns in either the human respiratory tract or the gastrointestinal tract Two different cell lines representing human lung (primary lung cells; ATCC #PCS-300-010) and human colorectal HRT-18G cells (ATCC #CRL-11663) were tested for cytotoxicity in March 2021. The methods, and results of these tests are tabulated in Table 1, above, and discussed below.

Methods:

These cells were cultured to 70-80% confluency using the ATCC recommended cell culture media (ATCC #PCS-300-030 for primary human lung, or ATCC #30-202 for colorectal cells). The cell culture medium was then removed and replaced with 100% concentration of the Goldshield LLC product provided. As a control for lack of appropriate growth media, 1× phosphate buffered saline (PBS) was added to control cells. These remained on the cells for 60 minutes. The cells were observed for any dead/dying cells floating in the culture media, or for any cells with altered morphology. At the end of the sixty minutes, the PBS or product was removed and replaced with regular cell growth media. The cells were again observed at 20 hours post-exposure for any detached cells or any with altered morphology. Experiments were performed in triplicate. Experiments were then repeated in triplicate to confirm the results of the first run.

Results:

No floating cells and no cells with altered morphology were detected in any of the replicates, from either round. The cells treated with the Goldshield LLC product were indistinguishable from the control cells treated with 1×PBS, a solution commonly used to wash cells which has no cytotoxic properties.

A 15% dilution of the Goldshield LLC product provided was also tested on these same cell types, and with the same protocol as above, just in case toxicity was observed. No toxicity was observed.

Conclusions:

The Goldshield LLC product provided does not induce any cytotoxic effects on the cell lines tested. GoldShield LLC may be interested in testing additional cell types in the future.

While the above is a description of what are presently believed to be the preferred embodiments of the invention, various alternatives, modifications, and equivalents may be used. Those skilled in the art will realize that other and farther embodiments can be made without departing from the spirit of the invention, and it is intended to include all such further modifications and changes as come within the true scope of the following claims. Therefore, the above description should not be taken as limiting the scope of the invention, which is defined solely by the claims.

I claim:

1. A nasal spray composition comprising:
a. an organosilane compound;
b. a botanical;
c. a steroid;
d. a polyol stabilizing agent; and
e. a nonionic surfactant;
wherein the composition adheres to mucosa, skin cells and hair in the nasal passages, creating a 12-24 hour bio-barrier to inhibit an infection from bacteria fungi, and viruses.

2. The nasal spray composition of claim 1, wherein the botanical is thymol.

3. The nasal spray composition of claim 1, wherein the steroid is dexamethasone.

4. The nasal spray composition of claim 1, wherein the composition reduces elements within the nasal passage by the use of the nonionic surfactant, permitting the adherence of the formula to goblet secretory cells, skin cells and hair.

5. The nasal spray composition of claim 1, wherein the organosilane linked to the botanical of the formula C₁₀H₁₄O and the steroid of the formula C₂₂H₂₉FO₅ adhere to the goblet secretory cells in the nasal passage and the composition begins the process of sanitizing and protecting these cells.

6. The nasal spray composition of claim 1, wherein the composition inhibits microbes for a period of at least 12-24 hours.

7. The nasal spray composition of claim 1, wherein the organosilane compound comprises an organosilane quaternary ammonium chloride and a botanical, Thymol.

8. The nasal spray composition of claim 1, wherein the organosilane is selected from a patented formula of Higgins and Shlisky, comprising a trihydroxysilyl propyldimethyl octadecyl ammonium chloride and a nonionic surfactant.

9. The nasal spray composition of claim 2, wherein the botanical, Thymol, is stabilized in water with a polyol, containing at least three hydroxy groups, any two of the three hydroxy groups separated by at least three intervening atoms.

10. The nasal spray composition of claim 1, wherein the polyol is selected from a group comprising; pentaerythritol, dipentaerythritol, tripentaerythritol, tetrapentaerythritol and tris(hydroxymethyl)ethane.

11. The nasal spray composition a of claim 1, wherein the nonionic surfactant is selected from a group composed of ethoxylated alcohols, ethoxylated nonyl phenol(s), and ethoxylated alkyl phenol(s).

12. The nasal spray composition of claim 1, wherein the botanical is Thymol with the formula C₁₀H₁₄O.

13. The nasal spray composition of claim 3, wherein the steroid, dexamethasone, is of the formula C₂₂H₂₉FO₅.

14. The nasal spray composition of claim 2, where the organosilane cross-links the carbon chains of the botanical, Thymol of the formula C₁₀H₁₄O.

15. The nasal spray composition of claim 3, wherein the organosilane cross-links the carbon chains of the steroid, dexamethasone, of the formula C₂₂H₂₉FO₅.

16. The nasal spray composition of claim 3, wherein the organosilane, botanical, Thymol and steroid retain a residual sanitizing function for at least 12-24 hours.

17. The nasal spray composition of claim 3, wherein the steroid, dexamethasone, is linked to the organosilane, and wherein said steroid accelerates the neutralization of the RNA's from both the proteins that the coronavirus uses to infect and invade cells.

18. The nasal spray composition of claim 3, wherein the composition is further includes additional medicinal agents, selected from the group consisting of dexamethasone, and numbing agent, a local anesthetic, including: menthol, hydrocodone, lidocaine, and tetrahydrozoline.

19. The nasal spray composition of claim 3, wherein the composition is formulated into oral or nasal sprays, aerosols, eye drops and solidified into lozenges.
